(19) United States
(12) Patent Application Publication
(10) Pub. No.: US 2022/0265591 A1
(43) Pub. Date: Aug. 25, 2022

(54) USE OF AN OSMOLYTE IN MANUFACTURE OF A MEDICAMENT FOR TREATMENT OF OCULAR DISORDERS

(71) Applicant: SINGAPORE HEALTH SERVICES PTELTD, Singapore (SG)

(72) Inventors: Jodhbir Singh MEHTA, Singapore (SG); Lakshminarayanan RAJAMANI, Singapore (SG); Anandalakshmi VENKATRAMAN, Singapore (SG)

(21) Appl. No.: 17/632,240

(22) PCT Filed: Aug. 3, 2020
(86) PCT No.: PCT/SG2020/050447

§ 371 Date: Feb. 2, 2022

(30) Foreign Application Priority Data
Aug. 2, 2019 (SG) 10201907137R

(51) Int. Cl. A61K 31/198; A61K 31/145; A61P 27/02

(52) U.S. Cl. CPC A61K 31/198; A61K 31/145; A61P 27/02

(57) ABSTRACT

The present disclosure provides a use of an osmolyte in the manufacture of a medicament treating protein aggregation related disorders, specifically provides a use of an osmolyte in the manufacture of a medicament treating TGFBI corneal dystrophies. The osmolyte is selected from a group consisting of betaine, raffmose, sarcosine, taurine and/or any pharmaceutically acceptable derivatives thereof.

### TECHNICAL FIELD

The present disclosure generally relates to a use of an osmolyte in the manufacture of a medicament treating protein aggregation related disorders, specifically relates to a use of an osmolyte in the manufacture of a medicament treating TGFBI corneal dystrophies.

### BACKGROUND

The cornea is a highly transparent and avascular tissue that forms the front part of the ocular surface. There are five different layers in human cornea, namely: epithelium, Bowman's membrane, stroma, Descemet's membrane and endothelium. Maintenance of corneal transparency is vital for vision. The collagen fibril in each layer of the cornea has a highly uniform diameter and the interfibrillar distances are also in a high degree of uniformity, which are the essential prerequisites for corneal transparency. Any defects or deformation to the above assembly can affect visual acuity.

Corneal dystrophies are a group of bilateral, symmetrical and heterogeneous inherited disorders leading to loss of corneal transparency and thereby to a reduction in visual acuity and in severe cases, blindness, which are characterized by the Age-dependent progressive deposition of misfolded proteins aggregates in various layers of the cornea. Mutations occurring in the transforming growth factor beta-induced (TGFBI) gene is the major cause of the majority of stromal corneal dystrophies (i.e., affecting the stroma).

Transforming growth factor beta-induced protein (TGFBIp) is a secreted 68 kDa extracellular matrix protein with 683 amino acids with four Fasciclin-like 1 (FAS1) domains, found in tissues throughout the body yet only the cornea is affected by the mutant proteins. Mutations in TGFBIp are not only known to alter the turnover rate but also alter the thermodynamic stability of the protein with several of the mutations leading to destabilized protein which is more likely to unfold18-20. The mutant protein also possesses different proteolytic processing and clearance mechanism in the eye compared to the wild type protein (WT). These proteolytic products can act as amyloid seeds that could trigger the TGFBIp aggregation pathway. To date, there are more than 65 reported mutations in the gene and 84% of the mutations are found in the fourth FAS1 domain making it a mutational hotspot. The mutant protein is associated with modified protein stability, altered proteolytic processing, and deposition of insoluble aggregates in various layers of the cornea. The aggregation and deposition of TGFBIp display different clinical phenotypes wherein the deposits range from amyloidogenic structures, amorphous granular deposits, or a combination thereof.

It has been reported that the peptides derived from the 1^{st} and 4^{th} FAS-1 domains of mutant TGFBIp have increased aggregation propensity compared to the WT. The protein composition of the amyloid fibrils from Lattice Corneal Dystrophy patients (LCD) has been disclosed by using mass spectrometric assays. The proteolytic fragments of TGFBIp derived from amyloid fibrils of the LCD patients, upon digestion with trypsin showed a higher abundance of peptides from the 4^{th} FAS-1 domain of TGFBIp compared to TGFBIp from healthy controls. Peptides G⁵¹¹DNRFSMLVAAIQSAGLTETLNR⁵³³, Y⁵⁷¹HIGDEILVSGGIGALVR⁵⁸⁸, E⁶¹¹PVAEPDIMATNGVVHVITNVLQPPANRPQER⁶⁴², and L⁴⁹⁷TPPMGTVMDVLKGDNRFSMLVAAIQSAGLTETLNR⁵³³ were found to be enriched in the patient samples compared to the controls.

Currently, the only available treatment of corneal dystrophies is surgical intervention, i.e., corneal transplantation or tissue replacement through surgery, such as Penetrating Keratoplasty (PK), Anterior Lamellar Keratoplasty (ALK) and the like, where the affected cornea is fully or partially replaced with a clear donor cornea by a surgeon. The major setback for patients with corneal dystrophies after surgery is the high recurrence of the disease with protein aggregation. Depending on the type of mutations in the TGFBI gene, the disease recurs within 5 years to 10 years. Besides, the current surgical intervention is cost-intensive, requires the effort and time of a trained conical surgeon and most importantly requires good quality donor corneal tissue.

With the rise in the ageing population, the increase in the report of patients with corneal dystrophy, the decrease in the number of good quality donor tissue, and the recurrence of protein aggregation even after surgery, there is a pressing need for simple, efficient and cost-effective novel treatments of corneal dystrophies.

### SUMMARY

To solve the problems mentioned above, the present disclosure provides a novel treatment of ocular disorders, specifically, provides the use of an osmolyte in the manufacture of a medicament for the treatment of ocular disorders.

According to some embodiments of the present disclosure, the osmolyte may be selected from a group consisting of betaine, raffinose, sarcosine, taurine and/or any pharmaceutically acceptable derivatives thereof.

According to some embodiments of the present disclosure, the osmolyte may be a combination of taurine and sarcosine.

According to some embodiments of the present disclosure, the osmolyte may be selected from a group consisting of taurine and/or any pharmaceutically acceptable derivatives thereof.

According to some embodiments of the present disclosure, the osmolyte may have a concentration of 0.01 mM to 1,000 mM.

According to some embodiments of the present disclosure, the osmolyte may have a concentration of 100 mM to 500 mM.

According to some embodiments of the present disclosure, the osmolyte may have a concentration of 200 mM and 320 mM.

According to some embodiments of the present disclosure, the medicament may be in a dosage form of drops, ointments, gels and/or injections.

According to some embodiments of the present disclosure, the medicament may be administrated for at least 24 hours to 12 months.

According to some embodiments of the present disclosure, the medicament may be administrated for at least 24 hours, 48 hours, 72 hours, 1 week, 2 weeks, 4 weeks, 2 months, 4 months, 6 months, 9 months or 12 months.

According to some embodiments of the present disclosure, the osmolyte may inhibit amyloid fibrillation and dissolve amyloid fibrils.

According to some embodiments of the present disclosure, the ocular disorders may be Transforming Growth Factor-Beta Induced (TGFBI) corneal dystrophies.

According to some embodiments of the present disclosure, the TGFBI corneal dystrophies may be Bowman's layer corneal dystrophies and stromal corneal dystrophies.

According to some embodiments of the present disclosure, the Bowman's layer corneal dystrophies may be Reis-Buckler corneal dystrophy (RBCD) and Thiel-Behnke corneal dystrophy (TBCD).

According to some embodiments of the present disclosure, the stromal comeal dystrophies may be lattice corneal dystrophies (LCD), Granular Corneal Dystrophies Type I (GCD1) and Type II (GCD2).

### BRIEF DESCRIPTION OF THE DRAWINGS

FIG. 1 shows the chemical structures of four osmolytes: betaine, raffinose, sarcosine and taurine, in accordance with embodiments of the present disclosure;

FIG. 2 shows the results of Thioflavin T (ThT) fluorescence assay on inhibition of amyloid fibrillation of TGFBIp⁶¹¹⁻⁶³³ G623R peptide by betaine, raffinose, sarcosine and taurine at various time points, respectively, in accordance with embodiments of the present disclosure;

FIG. 3A shows the ThT fluorescent microscopy images of the TGFBIp⁶¹¹⁻⁶³³ G623R peptides treated by betaine, raffmose, sarcosine and taurine, respectively, at various time points after treatment and FIG. 3B shows the quantitative analysis results of FIG. 3A, in accordance with embodiments of the present disclosure;

FIG. 4 shows the results of Circular Dichroism assays of the untreated TGFBIp⁶¹¹⁻⁶³³ G623R peptide and the peptide samples treated with betaine, raffmose, sarcosine and taurine, respectively, at 0 h (A), 24 h (B), 48 h (C) and 72 h (D) after treatment, in accordance with embodiments of the present disclosure;

FIG. 5 shows the results of ThT fluorescence assay on the dissolution of preformed amyloid fibrils from TGFBIp⁶¹¹⁻⁶³³ G623R peptide by betaine, raffmose, sarcosine and taurine, respectively, in accordance with embodiments of the present disclosure;

FIG. 6A shows the ThT fluorescent microscopy images of the preformed amyloid fibrils from TGFBIp⁶¹¹⁻⁶³³ G623R peptide treated with betaine, raffmose, sarcosine and taurine for 24 h, 48 hours and 72 hours, respectively and FIG. 6B shows the quantitative analysis results of FIG. 6A, in accordance with embodiments of the present disclosure;

FIG. 7 shows the results of Circular Dichroism assays for the preformed amyloid fibrils from TGFBIp⁶¹¹⁻⁶³³ G623R peptide: (A): the untreated preformed amyloid fibrils; the preformed amyloid fibril samples treated with betaine, raffinose, sarcosine and taurine, respectively, at 24 h (B), 48 h (C) and 72 h (D) after treatment, in accordance with embodiments of the present disclosure;

FIG. 8A shows the Scanning Electron Microscopy (SEM) images of the preformed amyloid fibrils from TGFBIp⁶¹¹⁻⁶³³ G623R peptide treated with betaine, raffinose, sarcosine and taurine for 72 h, respectively, in accordance with embodiments of the present disclosure;

FIG. 8B shows the Transmission Electron Microscopy (TEM) images of the preformed amyloid fibrils from TGFBIp⁶¹¹⁻⁶³³ G623R peptide treated with betaine, raffmose, sarcosine and taurine for 72 h, respectively, in accordance with embodiments of the present disclosure;

FIG. 9 shows the synergic effect of taurine and sarcosine on inhibition of amyloid fibrillation of TGFBIp⁶¹¹⁻⁶³³ G623R peptide: (A): ThT assay results; (B): CD assay results; and (C): fluorescence images, in accordance with embodiments of the present disclosure;

FIG. 10 shows the synergic effect of taurine and sarcosine on disaggregation of preformed amyloid fibrils from TGFBIp⁶¹¹⁻⁶³³ G623R peptide: (A): ThT assay results; (B): CD assay results; and (C): fluorescence images, in accordance with embodiments of the present disclosure;

FIG. 11 shows the inhibitory effect of taurine at different concentrations on TGFBIp⁶¹¹⁻⁶³³ G623R peptide: (A): ThT assay results; (B): CD assay results for 200 mM taurine; (C): CD assay results for 320 mM taurine; (D): fluorescence images, in accordance with embodiments of the present disclosure;

FIG. 12 shows the inhibitory effect of taurine at different concentrations on TGFBIp⁶¹¹⁻⁶³³ N622K peptide: (A): ThT assay results; (B): CD assay results for 200 mM taurine; and (C): CD assay results for 320 mM taurine, in accordance with embodiments of the present disclosure;

FIG. 13 shows the dissolution effect of taurine at different concentrations on the preformed amyloid fibrils from TGFBIp⁶¹¹⁻⁶³³ G623R peptide: (A): ThT assay results; (B): CD assay results for 200 mM taurine; (C): CD assay results for 320 mM taurine; and (D): fluorescence images, in accordance with embodiments of the present disclosure;

FIG. 14 shows the dissolution effect of taurine at different concentrations on the prefomied amyloid fibrils from TGFBIp⁶¹¹⁻⁶³³ N622K peptide: (A): ThT assay results; (B): CD assay results for 200 mM taurine; and (C): CD assay results for 320 mM taurine, in accordance with embodiments of the present disclosure;

FIG. 15 shows the SEM images of the preformed amyloid fibrils from TGFBIp⁶¹¹⁻⁶³³ G623R and TGFBIp⁶¹¹⁻⁶³³ N622K peptides treated with taurine at different concentrations, respectively, in accordance with embodiments of the present disclosure;

FIG. 16 shows the results of the cytotoxicity of osmolytes determined by MTT assays, in accordance with embodiments of the present disclosure; and

FIG. 17 shows live-cell images of HCSFs treated with varying concentrations of betaine, raffinose, sarcosine and taurine at various time points, in accordance with embodiments of the present disclosure.

### DETAILED DESCRIPTION

The present disclosure provides a novel treatment of corneal dystrophies that does not involve painful surgery, does not require donor tissue and is cost-effective. Specifically, the present disclosure provides the use of an osmolyte in the manufacture of a medicament for the treatment of ocular disorders, e.g., TGFBI corneal dystrophies.

Based on the mass spectrometric analysis of the peptide fragments of TGFBIp, the inventors characterized the amyloid-forming properties of the 23 amino acid long peptide (E⁶¹¹PVAEDIMATNGVVHVITNVLQ⁶³³) with amino acid substitutions that decreased the net charge of the peptides. This peptide region has been associated with more than 16 mutations that are clinically significant with higher potential to form amyloid fibrils even under physiological conditions. About 11 mutations in this peptide region are known to alter the overall net charge of TGFBIp. Based on the characterization of in-vitro aggregation properties of peptides, TGFBIp⁶¹¹⁻⁶³³ G623R and TGFBIp⁶¹¹⁻⁶³³ N622K were found to have a greater propensity to form amyloid fibrils. Besides, amyloid fibrils derived from peptide TGFBIp⁶¹¹⁻⁶³³ G623R displayed remarkable resistance to thermal denaturation when compared to WT fibrils.

In TGFBI corneal dystrophies, a mutant protein undergoes different proteolytic processing compared to the wild-type protein, which results in the production of short peptides that may act as amyloid aggregation seeds. Proteomic analysis of amyloid deposits from TGFBI corneal dystrophy patients showed enrichment of short peptides in the patient samples compared to the wild-type.

Osmolytes are small organic molecules, of diverse chemical structures, that regulate the solvent properties of cells, by conserving the native structures of proteins during an osmotic or thermal stress response. Osmolytes may be categorized as polyhydric alcohols, sugars (polyols), amino acids (and their derivatives), and methylammonium compounds. Osmolytes are widely used to stabilize and facilitate protein folding since they can act as “chemical chaperones”. Osmo-protectants and chemical chaperones have been shown to shift the equilibrium towards the native state, by exhibiting a thermodynamic stabilization of the protein. This is accomplished by repopulating the denatured and native states, via unfavorable interactions with protein surfaces (a combination of backbone and side-chain interactions). Hence, according to the present disclosure, osmolytes are being evaluated and asserted as modes of treatment in various protein aggregation related disorders like ocular disorders.

The present disclosure, as detailed hereinbelow, provides that use of an osmolyte inhibits amyloid fibrillation of the TGFBIp⁶¹¹⁻⁶³³ G623R peptides and of the TGFBIp⁶¹¹⁻⁶³³ N622K peptides, and further promotes dissolving and disaggregating of amyloid fibrils. And since synthesized TGFBIp peptides comprising amino acids 611-633 with G632R or N622K mutations from 4^{th} FAS1 domains of TGFBIp are most stable and highly amyloidogenic and the region encompasses 11 mutations that are clinically associated with Lattice Corneal Dystrophy (LCD) phenotype, thus, the model peptides TGFBIp⁶¹¹⁻⁶³³ G623R and TGFBIp⁶¹¹⁻⁶³³ N622K (Synpeptide Co Ltd, Shanghai, China) were used as in-vitro TGFBIp peptide aggregation models. Once the peptides cease to aggregate and accumulate in the cornea, due to use of osmolytes, ocular disorders that lead to visual acuity and even blindness by loss of corneal transparency, may be treated. Accordingly, the present disclosure provides use of an osmolyte in the manufacture of a medicament for the treatment of ocular disorders.

There have been few previously reported studies on TGFBIp model peptides and the effect of chemical compounds on amyloid fibril formation and treatment options to disaggregate amyloid fibrils. Kato et al. in “Benzalkonium chloride accelerates the formation of the amyloid fibrils of conical dystrophy-associated peptides”, J. Biol. Chem. 288(35), 25109-18. (2013), reported that the presence of preservatives such as benzalkonium chloride in eye drops close to the critical micellar concentration (0.001-0.02% (0.03-0.6 mM) accelerated the amyloid fibril formation in TGFBIp derived peptides and highlighted the potential risks associated with such formulations in eye drops. Use of several organic, polymeric, and inorganic nanoparticles, synthetic peptides, amino acids and sialic acid were reported to inhibit amyloid fibril formation. Palmal and Debnath et al., in “Inhibition of amyloid fibril growth and dissolution of amyloid fibrils by curcumin-gold nanoparticles”, Chemistry 20(20), 6184-91 (2014), reported the utility of curcumin conjugated gold nanoparticles and epigallactocatechin-3-gallate conjugated polymer nanoparticles for inhibition and dissolution of preformed amyloid fibrils of Aβ₁₋₄₀ peptide. The strong affinity of nanoparticles for both oligomeric and fibrillar form of peptides was suggested to be responsible for stabilization of soluble oligomers which in turn attenuated the neurotoxicity of β-oligomers. Unlike β-oligomers formed by Aβ₁₋₄₀ peptide, the soluble oligomers of TGFBIp 611-633 c.623 G>R were non-toxic. Thus, the dissolution of amyloid fibrils from TGFBIp by osmolytes was found hereinbelow to not have an adverse effect on the tissue and may be a useful therapeutic strategy for patients with corneal dystrophies, such that osmolytes may be used in the manufacture of a medicament for treating ocular disorders such as corneal dystrophies.

The present disclosure is the first to teach the use of non-cytotoxic osmolytes for the inhibition and disintegration of amyloid fibrils derived from TGFBI associated corneal dystrophies. There have been several previous attempts to generate a suitable transgenic animal model, either to knock-in or knock-out TGFBI gene, and to evaluate the pathologic role of the mutant protein. All the generated animal models were not very successful to express the disease phenotype or in the survival of the animals. Since there are more than 65 mutations reported in this disease, it will not be feasible to generate animal models that represent each mutation or a universal model that can be used to study all the mutant phenotypes. Hence, the in-vitro peptide aggregation model provided hereinbelow is more useful to study using osmolytes as part of a medicament that may be used to either prevent protein aggregation or dissolve preformed aggregates and thereby to treat ocular disorders.

In some embodiments of the present disclosure, it provides a use of an osmolyte in the manufacture of a medicament for the treatment of TGFBI corneal dystrophies, wherein the osmolyte can be selected from a group consisting of betaine, raffmose, sarcosine, taurine and/or any pharmaceutically acceptable derivatives thereof. The osmolytes contain numerous hydrogen bonding donors/acceptors which may interfere with the β-amyloid oligomers or fibrils.

In some other embodiments of the present disclosure, it provides a use of an osmolyte in the manufacture of a medicament for treatment of TGFBI corneal dystrophies, wherein the osmolyte can preferably be selected from a group consisting of any combinations of betaine, raffinose, sarcosine, taurine, preferably, can be a combination of taurine and sarcosine.

In yet other embodiments of the present disclosure, it provides a use of an osmolyte in the manufacture of a medicament for the treatment of TGFBI corneal dystrophies, wherein the osmolyte can preferably be selected from a group consisting of taurine and/or any pharmaceutically acceptable derivatives thereof.

In one embodiment of the present disclosure, it provides a use of an osmolyte in the manufacture of a medicament for the treatment of TGFBI corneal dystrophies, wherein the osmolyte can be taurine.

In some embodiments of the present disclosure, it provides a use of an osmolyte in the manufacture of a medicament for the treatment of TGFBI corneal dystrophies, wherein the osmolyte may have a concentration of 0.01 mM to 1,000 mM. Other osmolyte concentrations may be used.

In some other embodiments of the present disclosure, it provides a use of an osmolyte in the manufacture of a medicament for treatment TGFBI corneal dystrophies, wherein the osmolyte may have a concentration of 100 mM to 500 mM.

In yet other embodiments of the present disclosure, it provides a use of an osmolyte in the manufacture of a medicament for treatment TGFBI corneal dystrophies, wherein the osmolyte may have a concentration of 200 mM or 320 mM.

In some embodiments of the present disclosure, it provides a use of an osmolyte in the manufacture of a medicament for the treatment of TGFBI corneal dystrophies, wherein the medicament may be in a dosage form of drops, ointments, gels and/or injections.

In some embodiments of the present disclosure, it provides a use of an osmolyte in the manufacture of a medicament for the treatment of TGFBI corneal dystrophies, wherein the medicament can be administrated for at least 24 hours to 12 months.

In some embodiments of the present disclosure, it provides a use of an osmolyte in manufacture of a medicament for treatment of TGFBI corneal dystrophies, wherein the medicament can be administrated for at least 24 hours, 48 hours, 72 hours, 1 week, 2 weeks, 4 weeks, 2 months, 4 months, 6 months, 9 months or 12 months. Other administration periods may be implemented.

In some embodiments of the present disclosure, it provides a use of an osmolyte in the manufacture of a medicament for the treatment of TGFBI corneal dystrophies, wherein the osmolyte can inhibit amyloid fibrillation and dissolve amyloid fibrils.

Based on the anatomical location of the misfolded protein deposits, corneal dystrophies can be divided into two groups: Bowman's layer corneal dystrophies, such as Reis-Buckler corneal dystrophy (RBCD) and Thiel-Behnke corneal dystrophy (TBCD), or stromal corneal dystrophies, such as lattice corneal dystrophies (LCD), Granular Corneal Dystrophies Type I (GCD Type I or GCD1) and GCD Type II (GCD2).

In some embodiments of the present disclosure, it provides a use of an osmolyte in the manufacture of a medicament for the treatment of TGFBI corneal dystrophies, wherein the TGFBI conical dystrophies can be Bowman's layer corneal dystrophies and stromal corneal dystrophies.

In some other embodiments of the present disclosure, it provides a use of an osmolyte in the manufacture of a medicament for the treatment of TGFBI corneal dystrophies, wherein the Bowman's layer conical dystrophies can be Reis-Buckler corneal dystrophy (RBCD) and Thiel-Behnke corneal dystrophy (TBCD).

In some other embodiments of the present disclosure, it provides a use of an osmolyte in the manufacture of a medicament for the treatment of TGFBI corneal dystrophies, wherein the stromal conical dystrophies can be lattice corneal dystrophies (LCD), Granular Corneal Dystrophies Type I (GCD1) and Type II (GCD2).

In some other embodiments of the present disclosure, it provides a use of an osmolyte in the manufacture of a medicament for the treatment of TGFBI corneal dystrophies, wherein the TGFBI corneal dystrophies can have G632R mutation and/or N622K mutation in the 4^{th} FAS-1 domain of Transforming Growth Factor-Beta Induced Protein.

Before explaining at least one embodiment of the disclosure in detail, it is to be understood that the disclosure is not necessarily limited in its application to the details of construction and the arrangement of the components and/or methods set forth in the following description and/or illustrated in the drawings and/or the Examples. The disclosure is capable of other embodiments or of being practiced or carried out in various ways.

There is currently no animal model to mimic TGFBI corneal dystrophies phenotypes. Thus, in-vitro TGFBIp peptide aggregation models are used to investigate the effects of osmolytes on inhibiting peptide fibrillation and dissolving preformed amyloid fibrils. It has been found that the synthesized TGFBIp peptides comprising amino acids 611-633 with G632R or N622K mutations from 4^{th} FAS1 domains of TGFBIp are most stable and highly amyloidogenic and the region encompasses 11 mutations that are clinically associated with Lattice Corneal Dystrophy (LCD) phenotype. The synthetic TGFBIp peptides were 95% homogenous as confirmed by reversed-phase high-performance liquid chromatography.

In the present disclosure, the biological properties of a 23-residue peptide fragment that was highly abundant in the patient's cornea who carried the c.626 H>R mutation was compared. Decreasing the overall net charge of the 23-residue peptide (amino acid 611 to 633) by substitution with cationic residues resulted in a position-dependent alteration in the kinetics of amyloid formation26. Among these peptides, amyloid fibrils formed by TGFBIp 611-633 G623R peptide contained homogenous populations of 13-sheet assemblies, displayed excellent thermal stability when compared to other peptides. Thus, the model peptides TGFBIp⁶¹⁻⁶³³ G623R and TGFBIp⁶¹¹⁻⁶³³ N622K (Synpeptide Co Ltd, Shanghai, China) are used as in-vitro TGFBIp peptide aggregation models for assessing the efficacy of various osmolytes on amyloid inhibition as well as disaggregation efficiency.

Various biophysical, biochemical and microscopic methods such as Thioflavin T (ThT) fluorescence assay, Circular Dichroism spectroscopy and Scanning Electron Microscopy were employed to investigate the effect of osmolytes on amyloid fibrillation of the model peptides, including the ability of osmolytes to inhibit amyloid fibrillation of the peptides as well their ability to dissolve preformed amyloid fibrils formed by the peptides. Also, the cytotoxicity of osmolytes for Human Corneal Stromal Fibroblast (HCSFs) was further investigated.

### EXAMPLE 1

### Inhibitory Effect of Osmolytes on Amyloid Fibrillation

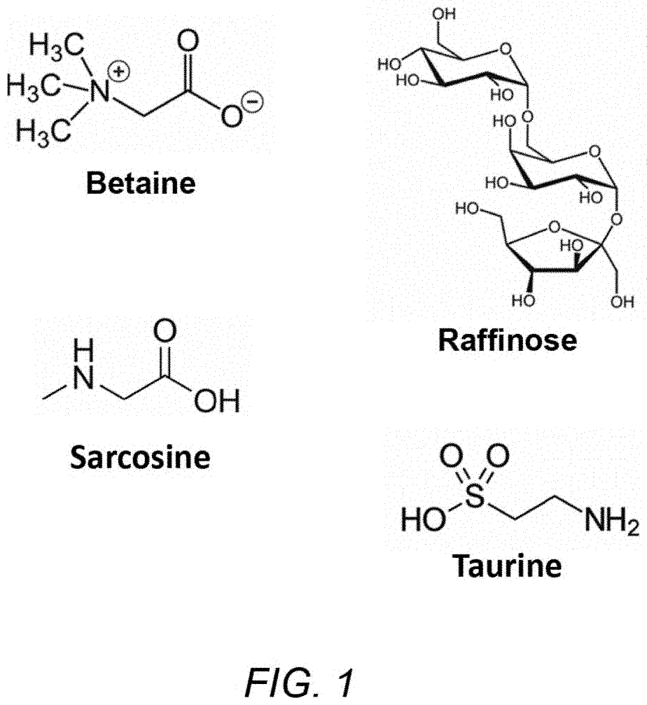

The 23-amino acid long peptide (TGFBIp 611-633 c.623 G>R) from the 4th FAS1 domain of TGFBIp with the substitution, G623→R (EPVAEPDIMATNRVVHVITNVLQ) that rapidly formed amyloid fibrils was used in this study. The peptide was dissolved (0.6 mg/ml) in PBS and allowed to form amyloid fibrils in 50 ml Falcon tubes in a shaking incubator at 37° C. and 180 rpm with and without the addition of osmolytes. To investigate the effect of osmolytes on the kinetics of amyloid fibrillation, the model peptide TGFBIp⁶¹¹⁻⁶³³ G623R was treated with 200 mM each of betaine, raffinose, sarcosine and taurine (Sigma-Aldrich Inc., MO, USA), which chemical structures are illustrated in FIG. 1, in 50 ml Falcon tubes for 24 hours (h), 48 hours and 72 hours, respectively. The TGFBIp⁶¹¹⁻⁶³³ G623R peptide incubated with PBS was used as a control.

Thioflavin T (ThT) assay:

The peptide samples collected at various time points above were treated with 30 μM Thioflavin T (ThT) (Sigma-Aldrich Inc., MO, USA) in PBS buffer at pH 5.5 in a 96-well microplate (Greiner Bio-One, Frickenhausen, Germany) for ThT fluorescence assay. The microplate was excited at 445 nm and the resulting emission fluorescence at 485 nm was measured using a microplate reader (Tecan Infinite M200 Pro, CA, USA).

Percentage inhibition for each osmolyte treatment was calculated by subtracting the baseline fluorescent intensity without the peptides and compounds from the observed fluorescent intensity of each treatment well. The fluorescent intensities per osmolyte treatment were normalized against the untreated fluorescent intensity per time point and expressed as a percentage.

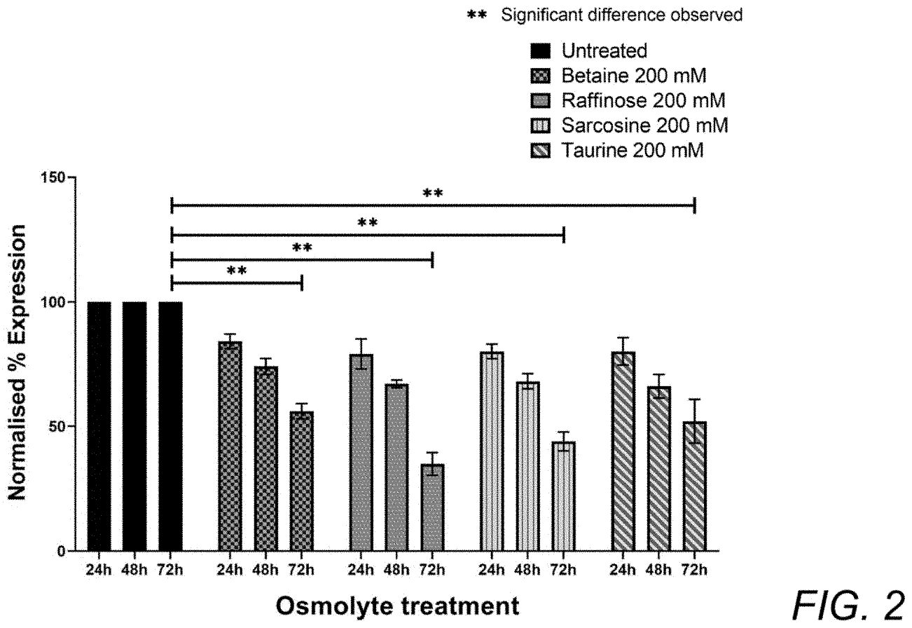

As shown in FIG. 2, the results of the ThT fluorescence assay indicated a time-dependent decrease in the ThT fluorescent intensity for the peptides treated with the four osmolytes when compared to the control-treated with PBS. When incubated alone, the peptide TGFBIp 611-633 G623R rapidly aggregated to form amyloid aggregates in 24 h, whereas incubation of peptides with an equimolar concentration of osmolytes resulted in a time-dependent decrease in amyloid formation. The effect was discernible even from 24 h post-treatment. The inhibitory effect was more significant at around 72 h, i.e. 65%±5 for raffinose, 56%±4 for sarcosine, 44%±3 for betaine and 48%±9 for famine (see Table 1(a)). The results shown in FIG. 2 demonstrate that all the four osmolytes inhibit amyloid fibril formation of the TGFBIp⁶¹¹⁻⁶³³ G623R peptide.

TABLE 1

| | | | |
|---|---|---|---|
| | | Treatment | % Inhibition |
| Osmolyte | Concentration | Time | (mean ± SD) n = 3 |

(a)

| | | | |
|---|---|---|---|
| Untreated (PBS) | | 24 h | 0 |
| | | 48 h | 0 |
| | | 72 h | 0 |
| Betaine | 200 mM | 24 h | 16 ± 3 |
| | | 48 h | 26 ± 3 |
| | | 72 h | 44 ± 3 |
| Raffinose | 200 mM | 24 h | 21 ± 6 |
| | | 48 h | 32 ± 2 |
| | | 72 h | 65 ± 5 |
| Sarcosine | 200 mM | 24 h | 20 ± 3 |
| | | 48 h | 32 ± 2 |
| | | 72 h | 56 ± 4 |
| Taurine | 200 mM | 24 h | 20 ± 6 |
| | | 48 h | 34 ± 5 |
| | | 72 h | 48 ± 9 |

(b)

| | | | |
|---|---|---|---|
| Betaine | 200 mM | 24 h | 8 ± 1 |
| | | 48 h | 19 ± 6 |
| | | 72 h | 34 ± 10 |
| Raffinose | 200 mM | 24 h | 19 ± 8 |
| | | 48 h | 23 ± 10 |
| | | 72 h | 57 ± 10 |
| Sarcosine | 200 mM | 24 h | 17 ± 6 |
| | | 48 h | 43 ± 10 |
| | | 72 h | 57 ± 8 |
| Taurine | 200 mM | 24 h | 25 ± 4 |
| | | 48 h | 54 ± 9 |
| | | 72 h | 56 ± 12 |

Table 1(a) illustrates inhibition of amyloid fibrillation of TGFBIp⁶¹¹⁻⁶³³ G623R peptide by osmolytes in ThT fluorescence assay. Table 1(b) illustrates comparison of θ[218] values of untreated and osmolyte-treated peptides at various time points determined by circular dichroism assays.

Fluorescence microscopy:

The inhibitory effects of the four osmolytes on amyloid fibrillation of the TGFBIp⁶¹¹⁻⁶³³ G623R peptide were investigated further by fluorescence microscopy after ThT staining. The peptide samples collected at various time points above were incubated ThT at a ratio of 1:1 ratio in the dark for 30 minutes to obtain solutions for fluorescence microscopy. 25 μl of the solutions were taken on to slides with coverslips and visualized under a fluorescent microscope (Axiolmager Z1, Carl Zeiss, Oberkochen, Germany).

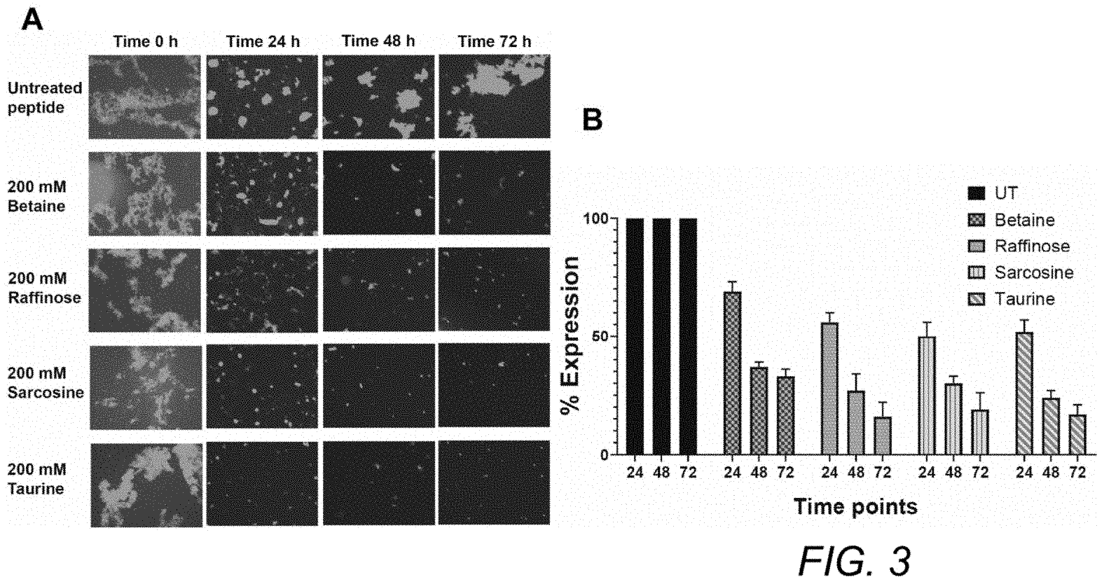

In FIG. 3A, the fluorescence images of the above samples treated with the osmolytes show smaller and fewer fluorescent spots compared to the control-treated with PBS. All osmolytes displayed inhibition of amyloid fibril formation of the peptide around 72 h. Especially, the fluorescence images of the above samples treated with taurine showed relatively smaller fluorescent spots even after 24 h compared to the samples treated with other osmolytes. The reduction in fluorescent intensity in the fluorescence images indicates fewer amyloid fibrils. In agreement with the ThT assay, the results shown in FIGS. 3A-3B clearly demonstrate that all the four osmolytes displayed the strong inhibitory effects on the amyloid fibrillation of the TGFBIp⁶¹¹⁻⁶³³ G623R peptide.

Circular Dichroism (CD) assay:

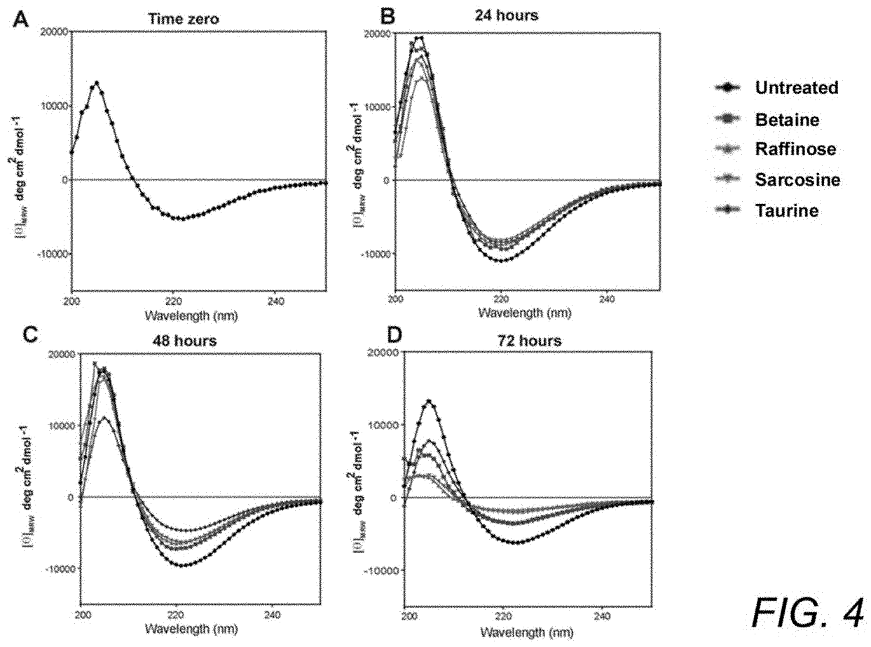

Circular Dichroism spectroscopy was performed to study the changes in the secondary structure of the peptides. The far UV-CD data for the TGFBIp⁶¹¹⁻⁶³³ G623R peptide without any osmolyte treatment after 24 h shows clear negative minima around 218 nm and a positive peak around 195 nm which are characteristics of β-sheet secondary structure (FIG. 4). This confirms that the native peptide has a high propensity to aggregate and form ordered structures within 24 h.

For peptide incubated with 200 mM Betaine, (FIG. 4) the ellipticity of peaks around 218 nm and 195 nm decreased progressively. The comparison of θ[218] value between the native peptide and 200 mM betaine treated showed 8%±1, 19%±6 and 34%±10 reduction (Table 1(b)) in the peak intensity at 24, 48 and 72 h respectively compared to the untreated sample. For peptide incubated with raffinose, a decreased intensity minima around 218 and a positive peak around 195 was still observed (FIG. 4). The θ[218] values show that the fibrillation of native peptide was inhibited by 19%±8, 23%±10 and 57%±10 at the 3 different time points respectively. Raffinose and Sarcosine showed maximum inhibition of TGFBIp 611-633 c.623 G>R peptide (FIG. 4). Visible characteristics of β-sheet amyloid fibrils were observed after 24 h and θ[218] value observed at three different time points showed about 17%±6, 43%±10 and 57%±8 inhibition of fibril formation when treated with sarcosine. With 200 mM taurine, the inhibition of fibrillation was observed with a reduction in characteristic β-sheet secondary structure and about 25%±4, 54%±9 and 56%±12 inhibition of fibrillation at the time points (FIG. 4).

The TGFBIp⁶¹¹⁻⁶³³ G623R peptides were treated with 200 mM betaine, raffinose, sarcosine and taurine in PBS buffer at pH 7.0 for 24 h, 48 h and 72 h, respectively. The peptide samples were collected 0.1 cm path length quartz cuvettes and examined in a Chirascan™-plus Spectropolarimeter (Applied Photophysics Limited, UK). Spectra were recorded from 260 nm to 190 nm in 0.1 nm steps at a scan rate of 50 nm/min. The final spectrum was the average of three scans as per the manufacturer's recommendation. The Mean Residual Weight (MRW) ellipticity ([θ]_{mrw} values) at wavelength λ was calculated using the following equation (i):

[θ]_{mrw}=MRW×θ_{λ}/10×l×c (i)

whereby θ_{λ} is the observed ellipticity at a particular wavelength (degrees), I is the path length (cm), and c is the concentration (g/ml). Secondary structures of the protein were analyzed using CDNN software.

The θ_{[218]} values that determine the β-sheet secondary structure were used to calculate the percentage inhibition in cross β-sheet in the untreated and osmolyte-treated peptide samples at various time points. Percentage inhibition for each osmolyte treatment per time point was calculated by normalizing the [θ]₂₁₈ values of osmolyte treatment with the [θ]₂₁₈ values of the untreated samples and expressed as a percentage.

The results of the Circular Dichroism assays were depicted in FIG. 4 and summarized in Table 1(b).

In the betaine treatment, the ellipticity peaks around 218 nm and 195 nm decreased progressively. The comparison of θ_{[218]} values between the native untreated peptide sample and the peptide sample treated with 200 mM betaine show that the peptide fibrillation was inhibited by 8%±1, 19%±6 and 34%±10 at 24 h, 48 h and 72 h, respectively, compared to the untreated sample.

For the peptide samples treated with raffmose, the presence of negative minima around 218 inn and a positive peak around 195 nm was still observed but with a greater decrease in CD intensity. The θ_{[218]} values show that the fibrillation of native peptide was inhibited by 19%±8, 23%±10 and 57%±10 at 24 h, 48 h and 72 h, respectively. Raffinose exhibited a maximum inhibitory effect ou fibrillation of the TGFBIp⁶¹¹⁻⁶³³ G623R peptide.

Sarcosine treatment showed an effect on inhibition of amyloid fibrillation of the peptide. Visible characteristics of β-sheet amyloid fibrils were observed after 24 h. The θ_{[218]} values showed that the peptide fibrillation was inhibited by about 17%±6, 43%±10 and 57%±8 at 24 h, 48 h and 72 h, respectively.

Taurine treatment showed an inhibitory effect on amyloid fibrillation of the peptide. The θ_{[}218] values showed that the peptide fibrillation was inhibited by about 25%±4, 54%±9 and 56%±12 at 24 h, 48 h and 72 h, respectively.

TABLE 2

| | | | |
|---|---|---|---|
| | | Treatment | % Inhibition |
| Osmolyte | Concentration | Time | (mean ± SD) n = 3 |

(a)

| | | | |
|---|---|---|---|
| Untreated (PBS) | | 24 h | 0 |
| | | 48 h | 0 |
| | | 72 h | 0 |
| Betaine | 200 mM | 24 h | 4 ± 3 |
| | | 48 h | 34 ± 11 |
| | | 72 h | 37 ± 17 |
| Raffinose | 200 mM | 24 h | 14 ± 4 |
| | | 48 h | 48 ± 6 |
| | | 72 h | 64 ± 8 |
| Sarcosine | 200 mM | 24 h | 24 ± 3 |
| | | 48 h | 47 ± 4 |
| | | 72 h | 53 ± 3 |
| Taurine | 200 mM | 24 h | 23 ± 1 |
| | | 48 h | 52 ± 5 |
| | | 72 h | 61 ± 2 |

(b)

| | | | |
|---|---|---|---|
| Betaine | 200 mM | 24 h | 6 ± 1 |
| | | 48 h | 24 ± 11 |
| | | 72 h | 36 ± 5 |
| Raffinose | 200 mM | 24 h | 7 ± 7 |
| | | 48 h | 48 ± 9 |
| | | 72 h | 58 ± 11 |
| Sarcosine | 200 mM | 24 h | 12 ± 8 |
| | | 48 h | 39 ± 6 |
| | | 72 h | 41 ± 2 |
| Taurine | 200 mM | 24 h | 13 ± 6 |
| | | 48 h | 40 ± 12 |
| | | 72 h | 54 ± 6 |

Table 2(a) illustrates dissolution of amyloid fibrils from TGFBIp⁶¹¹⁻⁶³³ G623R peptide by osmolytes studied with ThT fluorescence assay. Table 2(b) illustrates comparison of θ[218] values of untreated and osmolyte-treated preformed amyloid fibrils.

### EXAMPLE 2

### Effect of Osmolytes on Disaggregation of Preformed Amyloid Fibrils In Vitro Amyloid Fibrillation

The TGFBIp⁶¹¹⁻⁶³³ G623R peptides were incubated in PBS for 24 hours, allowed to form uniform amyloid fibrils. The TGFBIp⁶¹¹⁻⁶³³ G623R peptide solution was then subjected to Circular Dichroism spectroscopy to confirm the formation of amyloid fibrils. The result showed absorption minima around 218 nm and absorption maxima around 195 nm, which is very typical for a β-sheet rich secondary structure of amyloid fibrils. This suggests that the peptide formed amyloid fibrils within 24 h.

Thioflavin T (ThT) Assay:

The peptide solutions comprising preformed amyloid fibrils were treated with 200 mM each of betaine, raffinose, sarcosine and taurine in 50 ml Falcon tubes for 24 hours (h), 48 hours and 72 hours, respectively. The preformed amyloid fibril solution incubated with PBS was used as a control.

The peptide samples collected at the various time points above were treated with 30 μM ThT in PBS buffer at pH 5.5 in a 96-well microplate for ThT fluorescence assay. The microplate was excited at 445 mu and the resulting emission fluorescence at 485 nm was measured using a microplate reader (Tecan Infinite M200 Pro, CA, USA).

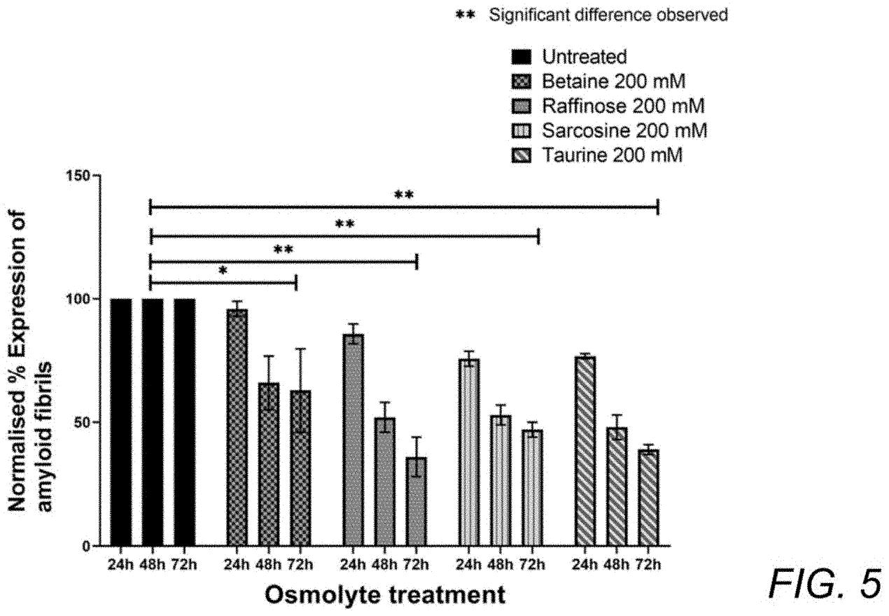

FIG. 5 shows that treatment of the preformed amyloid fibrils with osmolytes resulted in a time-dependent decrease in the ThT fluorescent intensities. Among the four osmolytes, raffinose and taurine treatment resulted in a significant decrease in ThT intensity by 64%±8 (raffinose) and 61%±2 (taurine), respectively, at 72 hours after treatment. The results as shown in FIG. 5 and Table 2 suggest that the osmolytes could disaggregate the preformed amyloid fibrils from the peptide, by disrupting non-covalent interactions of the β-sheet assembly.

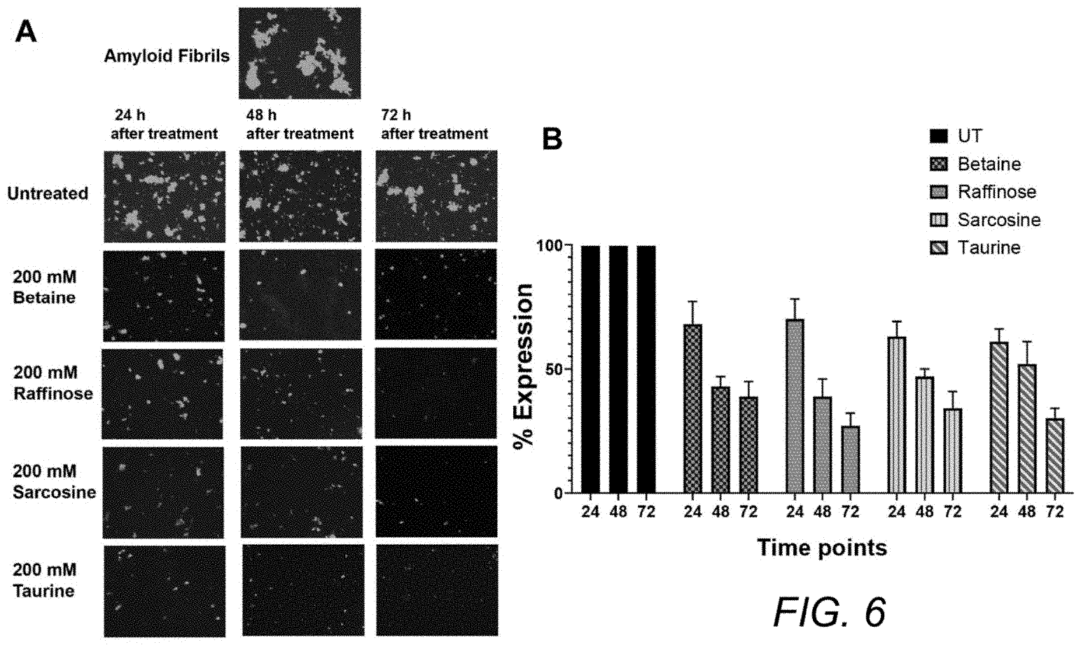

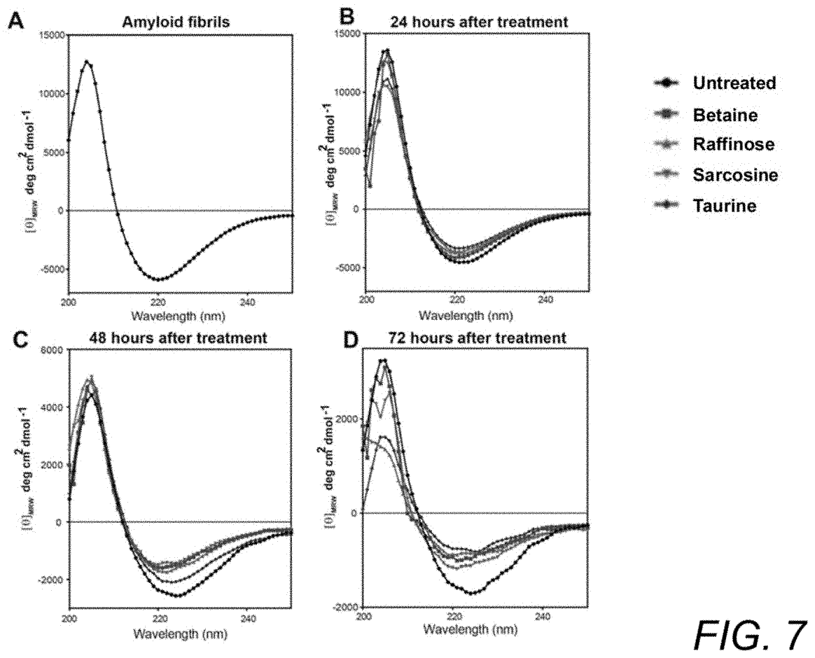

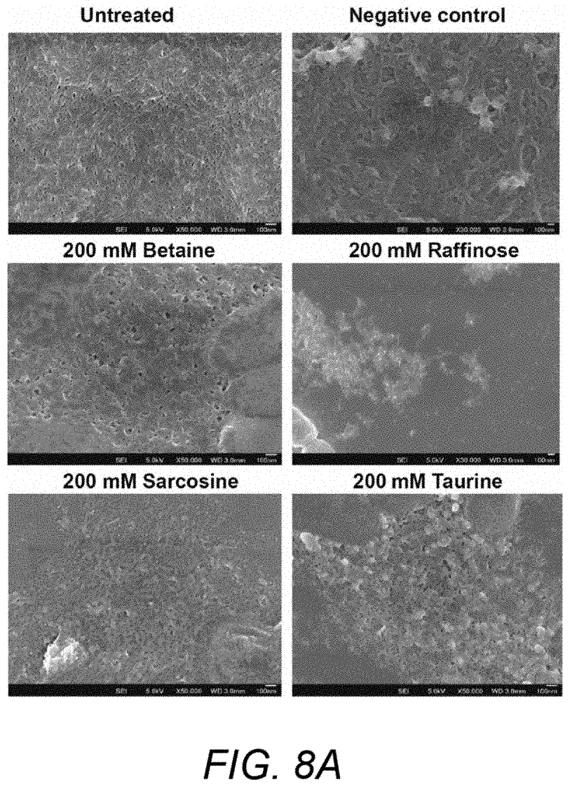

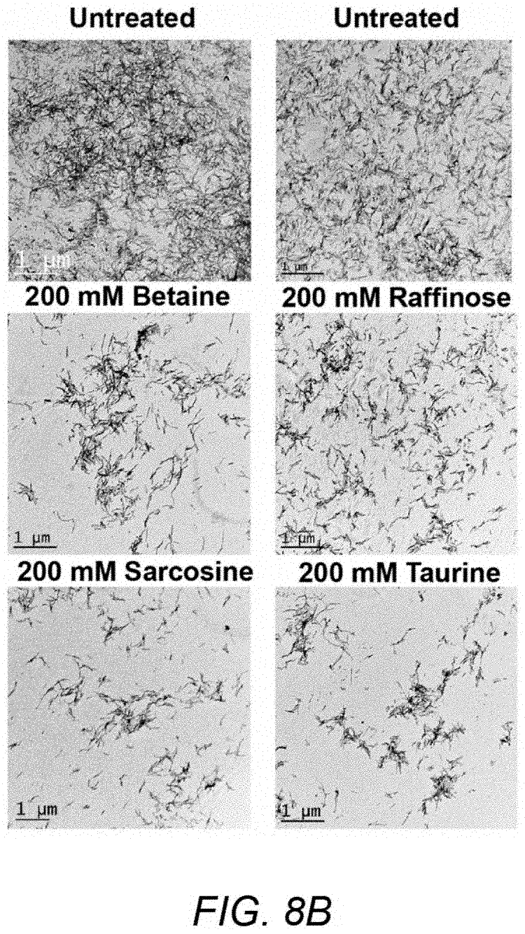

The preformed amyloid fibrils from the peptide were treated with osmolytes and followed up to 72 h post-treatment and visualized under the fluorescence microscope by adding ThT dye (FIG. 6). The results indicated a marked decrease in fluorescence staining for all the osmolytes after 24 h post-treatment. A progressive decrease in ThT staining was observed for all the osmolytes treated samples after 48 and 72 h post-treatment, suggesting disaggregation of the preformed amyloid fibrils. This clearly shows that all osmolytes effectively disaggregate preformed amyloid fibrils from TGFBIp⁶¹¹⁻⁶³³ G623R peptide. The disaggregation effect studied by far UV-CD experiments of native TGFBIp⁶¹¹⁻⁶³³ G623R peptide showed absorption minima around 218 um and absorption maxima around 195 nm (FIG. 7, Tables 1(b)-2(a)) which is very typical for a β-sheet rich secondary structure of amyloid fibrils. This shows that the peptide formed amyloid fibrils within 24 h, the presence of amyloid fibrils was confirmed by Transmission Electron Microscopy (TEM) analysis (FIG. 8B). Amyloid fibrils treated with osmolytes and followed after 72 h showed a marked decrease in the amplitude of the peak at [θ]₂₁₈. Among the different osmolytes Raffinose and Taurine showed around 58%±11 and 54%±6 dissolution respectively followed by sarcosine (41%±2) and betaine (36%±5) suggesting disruption of the preformed amyloid fibrils, and corroborate with the ThT results. Among the osmolytes, raffinose and tamine displayed the highest disruption of the β-sheet structure when compared to betaine and sarcosine.

Fluorescence Microscopy:

The ability of osmolytes to disaggregate the preformed amyloid fibrils were investigated further by fluorescence microscopy after ThT staining. The peptide samples collected at the various time points above were incubated at a ratio of 1:1 ratio of thpeptide solution to ThT in the dark for 30 minutes to obtain solutions for fluorescence microscopy. 25 μl of the solutions were taken on to slides with coverslips and visualized under a fluorescent microscope. Three representative images from each time point under a given condition was used for the quantitation of fluorescence. Image J software was used to quantify the signal from the images and the values for each time point per osmolyte treatment were normalised to the untreated sample at that particular time point.

As shown in FIGS. 6A-6B, the fluorescence images indicate a marked decrease in fluorescence staining for all the four osmolytes after 24 h post-treatment. A progressive decrease in ThT staining was observed for all the osmolyte-treated samples after 48 h and 72 h post-treatment, suggesting disaggregation of the preformed amyloid fibrils. This clearly shows that all the four osmolytes effectively disaggregate the preformed amyloid fibrils from the TGFBIp⁶¹¹⁻⁶³³ G623R peptide.

Circular Dichroism (CD) Assay:

Circular Dichroism (CD) spectroscopy was performed to study the disaggregation of the preformed amyloid fibrils. The preformed amyloid fibrils from the TGFBIp⁶¹¹⁻⁶³³ G623R peptides were treated with 200 mM each of betaine, raffmose, sarcosine and taurine in PBS buffer at pH 7.0 for 24 h, 48 h and 72 h, respectively. The treatment solutions were studied by far UV-CD assay.

The results of the CD assay (FIG. 7 and Table 2(b)) showed a marked decrease in the amplitude of the peak at [θ]₂₁₈, which suggests disruption and disaggregation of the preformed amyloid fibrils. Among the four osmolytes, raffinose and taurine displayed the highest disruption of the β-sheet structure of the amyloid fibrils compared to betaine and sarcosine.

Scanning Electron Microscopy (SEM):

Scanning Electron Microscopy (SEM, FEI-QUANTA 200F, The Netherlands) was performed to analyze the morphology of the TGFBIp⁶¹¹⁻⁶³³ G623R fibrils to verify the disaggregation of the preformed amyloid fibrils at 72 h after osmolytes treatment.

As shown in FIG. 8, the long bundle like morphology, typical of amyloid fibrils was clearly visible for the untreated native TGFBIp⁶¹¹⁻⁶³³ G623R peptide. When the amyloid fibrils were treated with betaine, sarcosine and taurine, the reduction in density of ordered amyloid fibrils was evident. When treated with raffmose, the SEM image showed disaggregation of amyloid fibrils with the absence of densely packed amyloid fibrils. The SEM results demonstrate that all the four osmolytes have great propensity to disaggregate a stable amyloid fibril structure. Among the four osmolytes, it was observed that raffmose had the maximum disaggregation propensity, followed by taurine, sarcosine and betaine.

Transmission Electron Microscopy (TEM):

The disaggregation of preformed amyloid fibrils was also verified by TEM analysis as illustrated in FIG. 8B. Morphological analysis of the TGFBIp 611-633 c.623 G>R fibrils before and after treatment with osmolytes for 72 hours was investigated by Transmission electron microscopy (TEM) with a JEOL JEM-1010 transmission electron microscope using Digital Micrograph™ 1.81.78 for GMS 1.8.0 (Gatan, Pleasanton, Calif.) at the Singhealth-core Electron Microscopy facility. 10 μl of amyloid fibril samples with and without osmolyte treatment were applied onto Formvar-carbon coated 300-mesh-size nickel grids. The samples were stained with Uranyl acetate solution, washed, dried, and observed at magnifications 8000-50000× at 80 kV.

The long bundle like morphology, typical of amyloid fibrils was visible for the untreated native TGFBIp⁶¹¹⁻⁶³³ G623R peptide. Addition of osmolytes to the amyloid aggregates showed disaggregation of amyloid fibrils with the absence of densely packed amyloid fibrils. The reduction in density of ordered amyloid fibrils was evident when the amyloid fibrils were treated with raffmose, betaine, sarcosine and taurine. The results are also in agreement with the ThT fluorescence, circular dichroism and immunofluorescence imaging.

### EXAMPLE 3

### Synergic Effect of Taurine and Sarcosine on Inhibition of Amyloid Fibrillation and Disaggregation of Preformed Amyloid Fibrils

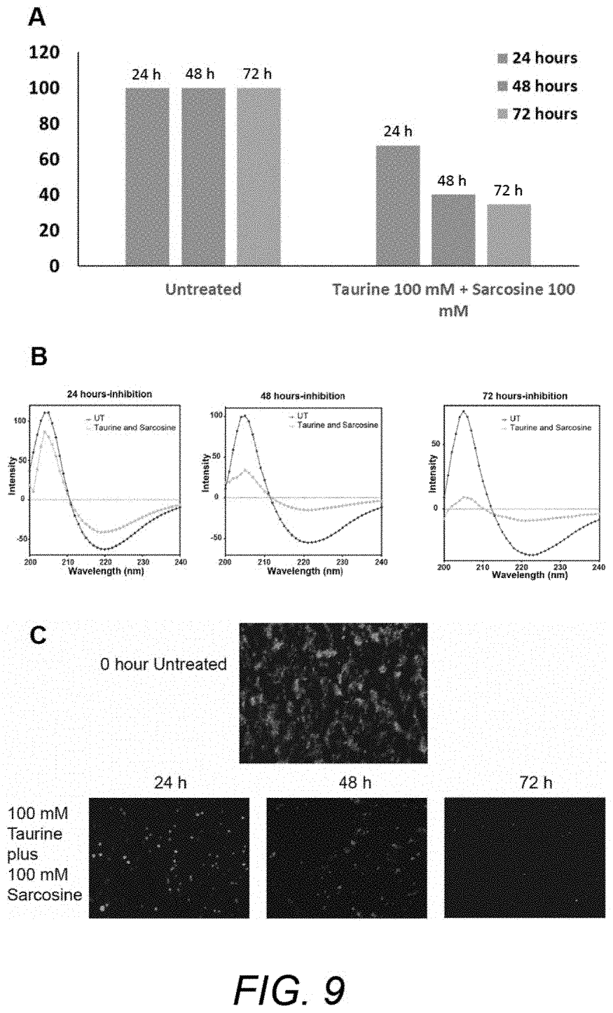

To investigate the synergic effect of taurine and sarcosine on inhibition of amyloid fibrillation, the model peptide TGFBIp⁶¹¹⁻⁶³³ G623R was treated with 100 mM of taurine and 100 mM of sarcosine (Sigma-Aldrich Inc., MO, USA) in 50 ml Falcon tubes for 24 hours (h), 48 hours and 72 hours, respectively, the results of which are illustrated in FIG. 9.

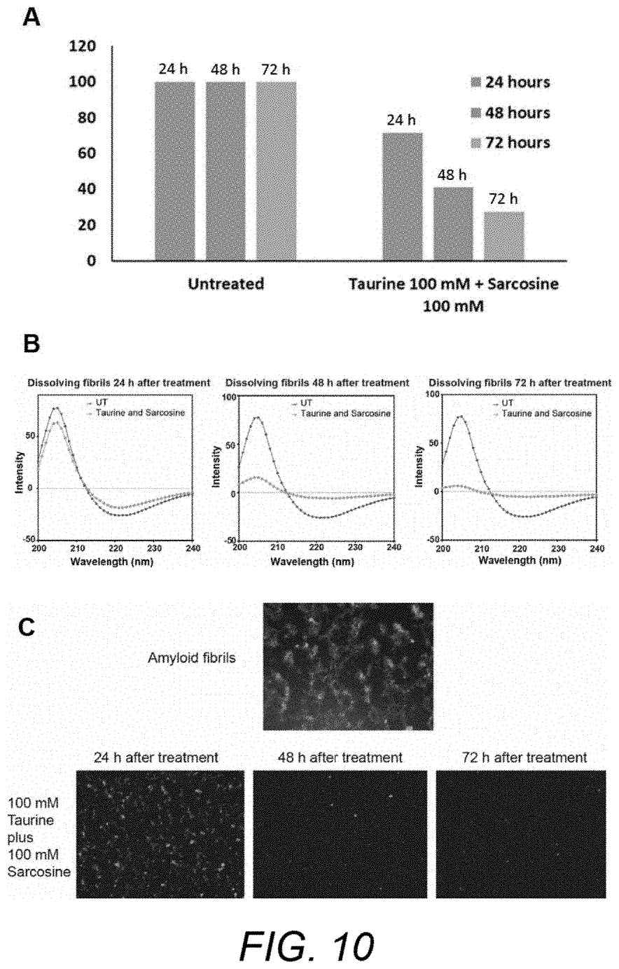

To investigate the synergic effect of taurine and sarcosine on disaggregation of preformed amyloid fibrils, the preformed amyloid fibrils from the model peptide TGFBIp⁶¹¹⁻⁶³³ G623R was treated with 100 mM of taurine and 100 mM of sarcosine (Sigma-Aldrich Inc., MO, USA) in 50 ml Falcon tubes for 24 hours (h), 48 hours and 72 hours, respectively, the results of which are illustrated in FIG. 10.

Thioflavin T (ThT) fluorescence assay, Circular Dichroism (CD) assay and ThT fluorescent microscopy were performed on the samples that were collected at various designated time points, respectively.

The results shown in FIGS. 9 and 10 and Table 3 suggest that the treatments with taurine and sarcosine result in higher inhibition/dissolution percentage that the treatments with taurine or sarcosine alone, especially from 48 hours after treatment, which means that taurine and sarcosine has synergic effects on inhibiting amyloid fibrils.

TABLE 3
Synergic effects of taurine and sarcosine on inhibition/disaggregation
in ThT fluorescence assay

| | | | % Inhibition/ |
|---|---|---|---|
| Effect | Assay | Treatment Time | Dissolution |
| Inhibition | ThT | 24 h | 33% |
| | ThT | 48 h | 62% |
| | ThT | 72 h | 65% |
| | CD | 24 h | 34% |
| | CD | 48 h | 73% |
| | CD | 72 h | 74% |
| Dissolution | ThT | 24 h | 29% |
| | ThT | 48 h | 59% |
| | ThT | 72 h | 73% |
| | CD | 24 h | 32% |
| | CD | 48 h | 55% |
| | CD | 72 h | 56% |

### EXAMPLE 4

### Inhibitory Effect of Taurine at Different Concentrations on Amyloid Fibrillation

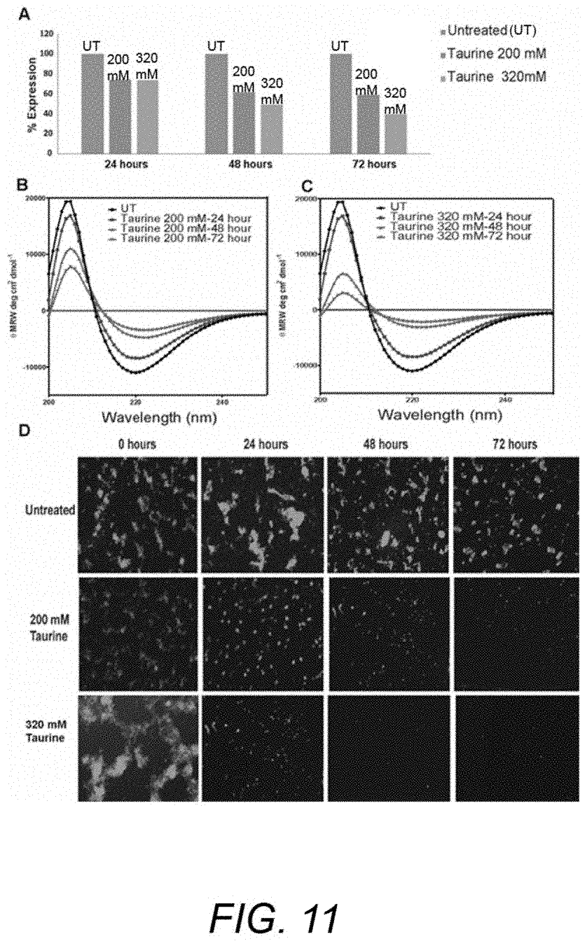

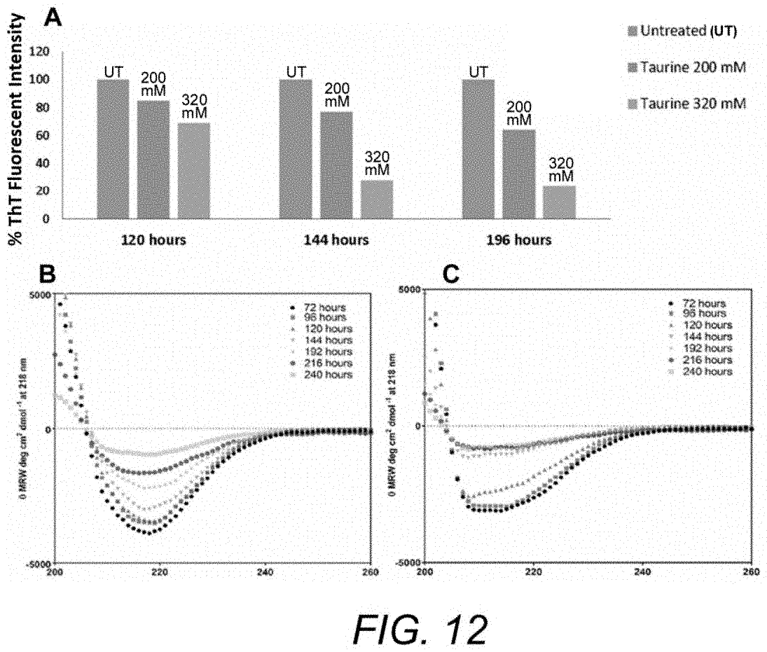

The TGFBIp⁶¹¹⁻⁶³³ G623R peptides were treated with 200 mM and 320 mM of taurine for up to 72 hours, respectively, the results of which are illustrated in FIG. 11. The TGFBIp⁶¹¹⁻⁶³³ N622K peptides were treated with 200 mM and 320 mM of taurine for up to 240 hours, respectively, the results of which are illustrated in FIG. 12. The peptides incubated with PBS were used as controls.

Thioflavin T (ThT) Assay

The peptide samples were collected at 24 h, 48 h and 72 h after treatment for TGFBIp⁶¹¹⁻⁶³³ G623R peptides and at 120 h, 144 h and 196 h after treatment for the TGFBIp⁶¹¹⁻⁶³³ N622K peptides. The samples were then treated with 30 μM ThT in PBS buffer at pH 5.5 in a 96-well microplate for ThT fluorescence assay. The microplate was excited at 445 nm and the resulting emission fluorescence at 485 nm was measured using a microplate reader.

In FIG. 11A and Table 4, the ThT assays results showed the inhibitory effects of 200 mM and 320 mM Taurine on the TGFBIp⁶¹¹⁻⁶³³ G623R peptide. Taurine was very efficient to inhibit amyloid fibrillation by 41% and 61% respectively for both concentrations.

In FIG. 12A and Table 4, the ThT assays results showed the dissolution effects of 200 mM and 320 mM Taurine on the TGFBIp⁶¹¹⁻⁶³³ N622K peptide. Taurine inhibited amyloid fibrillation by 37% and 76% respectively for both concentrations.

Circular Dichroism (CD) Assays

Circular Dichroism (CD) spectroscopy was performed to study the changes in the secondary structure of the peptides. The TGFBIp⁶¹¹⁻⁶³³ G623R peptides were treated with 200 mM and 320 mM of taurine in PBS buffer at pH 7.0 for 24 h, 48 h and 72 h, respectively. While the TGFBIp⁶¹¹⁻⁶³³ N622K peptides were treated with 200 mM and 320 mM of taurine in PBS buffer at pH 7.0 for 72 h, 96 h, 120 h, 144 h, 192 h, 216 h, and 240 h, respectively. The treatment solutions were studied by far UV-CD assay.

The CD assay results showed the inhibitory effect of 200 mM taurine (FIG. 11B) and 320 mM taurine (FIG. 11C) on the TGFBIp⁶¹¹⁻⁶³³ G623R peptide and 200 mM taurine (FIG. 12B) and 320 mM taurine (FIG. 12C) on the TGFBIp⁶¹¹⁻⁶³³ N622K peptide. Taurine was very efficient to inhibit amyloid fibrillation by 41% and 61% for the TGFBIp⁶¹¹⁻⁶³³ G623R peptide and by 39% and 66% for the TGFBIp⁶¹¹⁻⁶³³ N622K peptide, respectively for both concentrations.

Fluorescence Microscopy

The inhibition effect of taurine on the amyloid fibrillation was investigated further by fluorescence microscopy after ThT staining. The samples collected at the various time points above were incubated ThT at a ratio of 1:1 ratio in the dark for 30 minutes to obtain solutions for fluorescence microscopy. 25 μl of the solutions were taken on to slides with coverslips and visualized under a fluorescent microscope.

As shown in FIG. 11D, the TGFBIp⁶¹¹⁻⁶³³ G623R peptide treated with 200 mM and 320 mM of taurine show a reduction in fluorescent intensity when viewed under a microscope indicating the absence of amyloid

The results of the ThT assays, CD assays and fluorescence microscopy for the TGFBIp⁶¹¹⁻⁶³³ G623R and TGFBIp⁶¹¹⁻⁶³³ N622K peptides were also summarized in Table 4.

TABLE 4
Inhibition effects of taurine at different
concentrations on model peptides

| | | Taurine | % Inhibition |
|---|---|---|---|
| Peptide | Assay | concentration | at final time point |
| TGFBIp-G623R | ThT | 200 mM | 41% |
| | ThT | 320 mM | 60% |
| | CD | 200 mM | 42% |
| | CD | 320 mM | 63% |
| TGFBIp-N622K | ThT | 200 mM | 37% |
| | ThT | 320 mM | 76% |
| | CD | 200 mM | 39% |
| | CD | 320 mM | 66% |

### EXAMPLE 5

### Dissolution Effect of Taurine at Different Concentrations on Amyloid Fibrils

In Vitro Amyloid Fibrillation

The model peptides TGFBIp⁶¹¹⁻⁶³³ G623R and TGFBIp⁶¹¹⁻⁶³³ N622K were incubated in PBS for 24 hours for the TGFBIp⁶¹¹⁻⁶³³ G623R peptide and 96 hours for the TGFBIp⁶¹¹⁻⁶³³ N622K peptide, allowed to form uniform amyloid fibrils. The formation of amyloid fibrils was confirmed by (SEM) and CD spectroscopy.

Thioflavin T (ThT) Assay

The preformed amyloid fibrils were treated with taurine at a concentration of 200 mM and 320 mM for up to 72 hours, respectively. The preformed amyloid fibrils incubated with PBS were used as controls.

The samples were collected at 24 h, 48 h and 72 h after treatment for the TGFBIp⁶¹¹⁻⁶³³ G623R peptides, and at 24 h, 48 h, 96 h and 144 h after treatment for the TGFBIp⁶¹¹⁻⁶³³ N622K peptides. The samples were then treated with 30 μM ThT in PBS buffer at pH 5.5 in a 96-well microplate for ThT fluorescence assay. The microplate was excited at 445 nm and the resulting emission fluorescence at 485 nm was measured using a microplate reader.

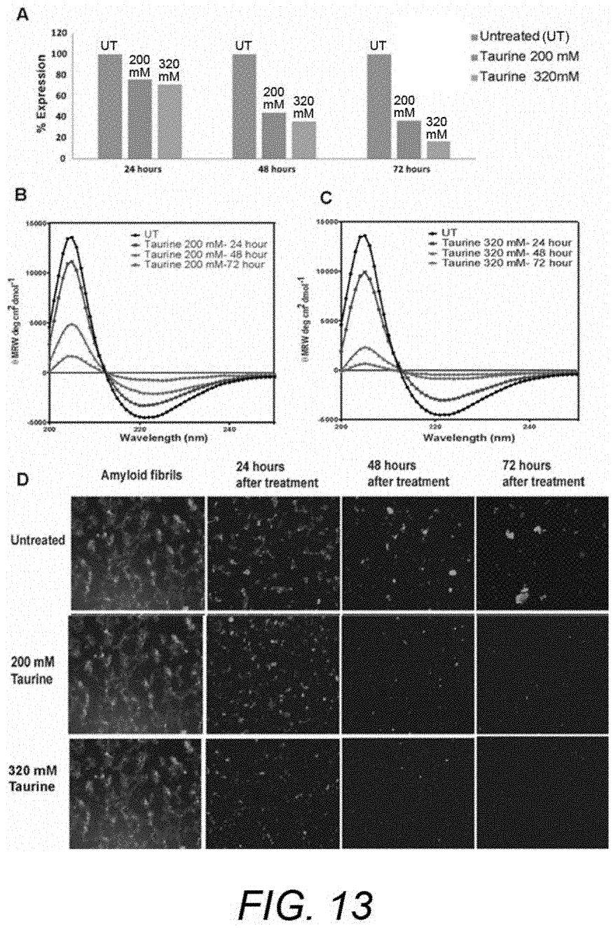

In FIG. 13A and Table 5, the ThT assays results showed the dissolution effects of 200 mM and 320 mM Taurine on amyloid fibrils from the TGFBIp⁶¹¹⁻⁶³³ G623R peptide. Taurine was very efficient to dissolve amyloid fibrils by 63% and 83% respectively for both concentrations.

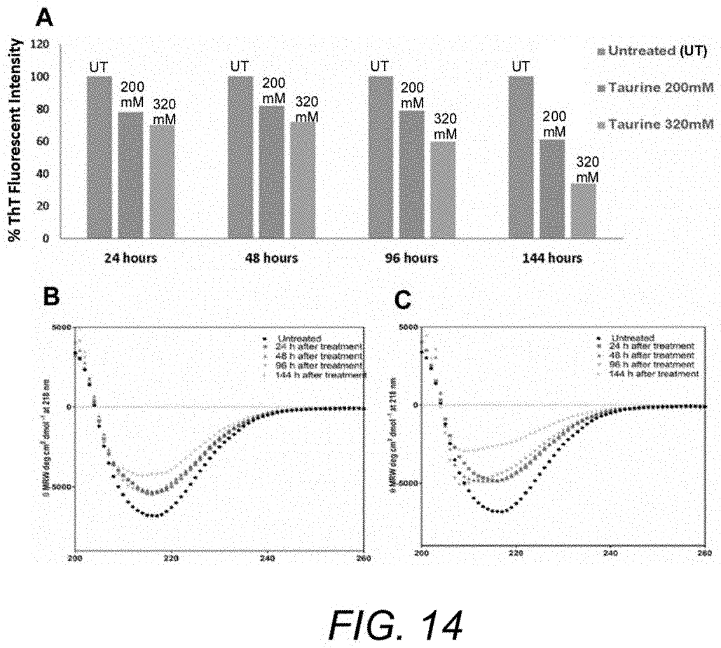

In FIG. 14A, the ThT assays results showed the dissolution effects of 200 mM and 320 mM Taurine on amyloid fibrils from the TGFBIp⁶¹¹⁻⁶³³ N622K peptide. Taurine was very efficient to dissolve amyloid fibrils by 83% and 88% respectively for both concentrations.

Circular Dichroism (CD) assays

Circular Dichroism (CD) spectroscopy was performed to study the disaggregation of the preformed amyloid fibrils. The preformed amyloid fibrils were treated with taurine at a concentration of 200 mM and 320 mM in PBS buffer at pH 7.0 for 24 h, 48 h and 72 respectively. The treatment solutions were studied by far UV-CD assay.

The CD assay results showed dissolution effect of 200 mM taurine (FIG. 13B) and 320 mM taurine (FIG. 13C) on the preformed amyloid fibrils from the TGFBIp⁶¹¹⁻⁶³³ G623R peptide, and 200 mM taurine (FIG. 14B) 320 mM taurine (FIG. 14C) on the TGFBIp⁶¹¹⁻⁶³³ N622K peptide. Taurine at both concentrations showed a significant reduction in β-sheet formation, which is the classical secondary structure of amyloid fibrillation process. The secondary structures for the TGFBIp⁶¹¹⁻⁶³³ G623R peptides were reduced by 49% and 69% by 200 mM and 320 mM of taurine, respectively. While the secondary structures for the TGFBIp N622K peptides were reduced by 61% and 66% by 200 mM and 320 mM of taurine, respectively.

Fluorescence Microscopy

The inhibition effect of taurine on the preformed amyloid fibrils was investigated further by fluorescence microscopy after ThT staining. The samples collected at the various time points above were incubated ThT at a ratio of 1:1 ratio in the dark for 30 minutes to obtain solutions for fluorescence microscopy. 25 μl of the solutions were taken on to slides with coverslips and visualized under a fluorescent microscope.

As shown in FIG. 13D, the preformed TGFBIp⁶¹¹⁻⁶³³ G623R fibrils treated with 200 mM and 320 mM of taurine show a reduction in fluorescent intensity when viewed under a microscope indicating the absence of amyloid fibrils.

The results of the ThT assays, CD assays and fluorescence microscopy for the TGFBIp⁶¹¹⁻⁶³³ G623R and TGFBIp⁶¹¹⁻⁶³³ N622K peptides were also summarized in Table 5.

TABLE 5
Dissolution effects of taurine at different
concentrations on model peptides

| | | Taurine | % Dissolution |
|---|---|---|---|
| Peptide | Assay | concentration | at final time point |
| TGFBIp-G623R | ThT | 200 mM | 64% |
| | ThT | 320 mM | 83% |
| | CD | 200 mM | 49% |
| | CD | 320 mM | 69% |
| TGFBIp-N622K | ThT | 200 mM | 83% |
| | ThT | 320 mM | 88% |
| | CD | 200 mM | 61% |
| | CD | 320 mM | 66% |

Scanning Electron Microscopy (SEM)

SEM was performed to analyze the morphology of the TGFBIp⁶¹¹⁻⁶³³ G623R and TGFBIp⁶¹¹⁻⁶³³ N622K fibrils to verify the disaggregation of the preformed amyloid fibrils at 72 h after osmolytes treatment.

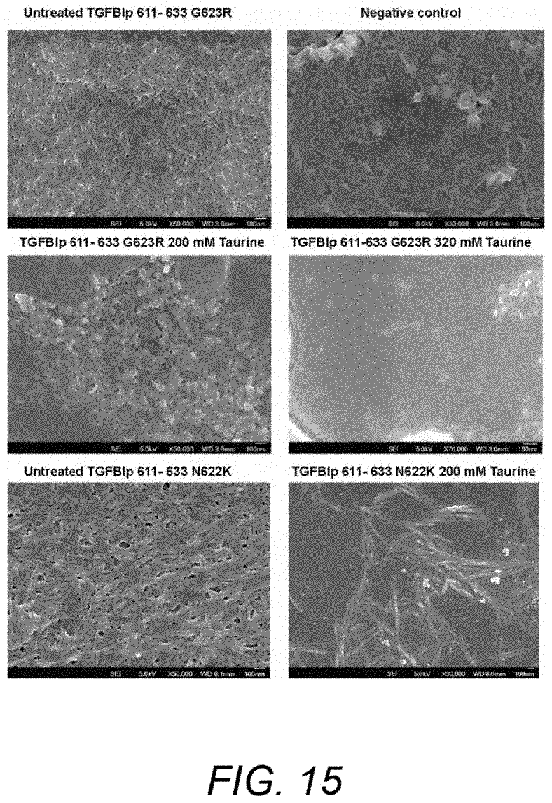

As shown in FIG. 15, when the amyloid fibrils were treated with and taurine, the reduction in density of ordered amyloid fibrils was evident. Especially, when the TGFBIp⁶¹¹⁻⁶³³ N622K fibrils were treated with 320 mM taurine, the SEM images showed disaggregation of amyloid fibrils with the absence of densely packed amyloid fibrils.

Conclusion

The results suggest that taurine performed efficiently to inhibit and dissolve amyloid fibrils derived from the model peptides with most TGFBI mutations.

### EXAMPLE 6

### Cytotoxicity Assay of Osmolytes

Cell Viability by MTT Assay

To assess the cytotoxic effect of osmolytes, MTT (3-(4,5-Dimethylthiazol-2-Y1)-2,5-Diphenyltetrazolium Bromide) assay was performed to study the cell viability of osmolyte-treated Human Comeal Stromal Fibroblast (HCSFs). The HCSFs were exposed to 0.1 mM, 1 mM, 10 mM, 100 mM and 1,000 mM of betaine, raffmose, sarcosine and taurine, respectively. The HCSFs treated with PBS was used as a control.

The media of the HCSF cultures were replaced with 100 μl of PBS(+) followed by the addition of 10 μl of 12 mM MTT solution Vybrant® MTT Cell Proliferation Assay Kit (Life Technologies, #V13154). The HCSF cells were incubated in the dark at 37° C. for 3 hours before dissolving of formazan crystals with DMSO through the removal of all but 25 μl of medium and adding 50 μof DMSO followed by thorough mixing and incubation at 37 ° C. for 10 minutes. Then the samples for the assay were obtained. The absorbance of each sample was taken at 540 nm with a microplate reader (Tecan Infinite® 200 PRO).

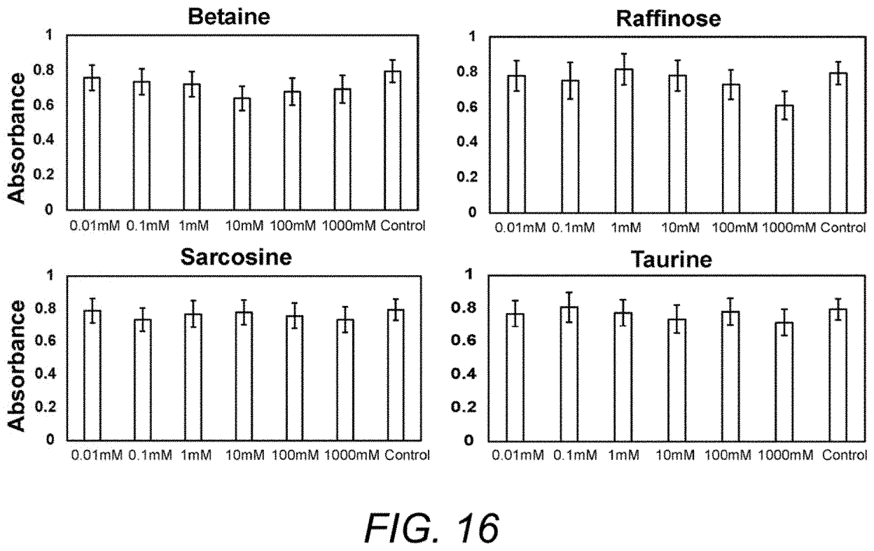

The observed absorbance values at different concentrations of osmolytes were subjected to statistical analysis. The statistical results shown in FIG. 16 did not show any statistical significance between the HCSFs treated with various concentrations of osmolytes and the PBS treated controls, which suggests that the four osmolytes at the indicated concentrations even at the elevated concentrations (1 M) are non-cytotoxic to the HCSFs and do not have an inhibitory effect on the proliferation of HCSFs.

Real-Time Live-Cell Imaging by IncuCyte

For real-time observation of the effect of the osmolytes on HCSFs, 3,000 cells/well were seeded in 96-well plates and placed in an incubator at 37° C. for 24 hours to proliferate. Cells were then incubated with 0.1 mM, 1 mM, 10 mM, 100 mM and 1,000 mM of betaine, raffinose, sarcosine and taurine, respectively, in triplicates. Images of cells were captured with IncuCyte ZOOM® System (Essen BioScience Inc., Research Instruments, Singapore) before and after addition of the osmolytes. Frames were then captured at 4-h intervals from 4 separate regions/well using a 10× objective for 20 h to observe cell toxicity.

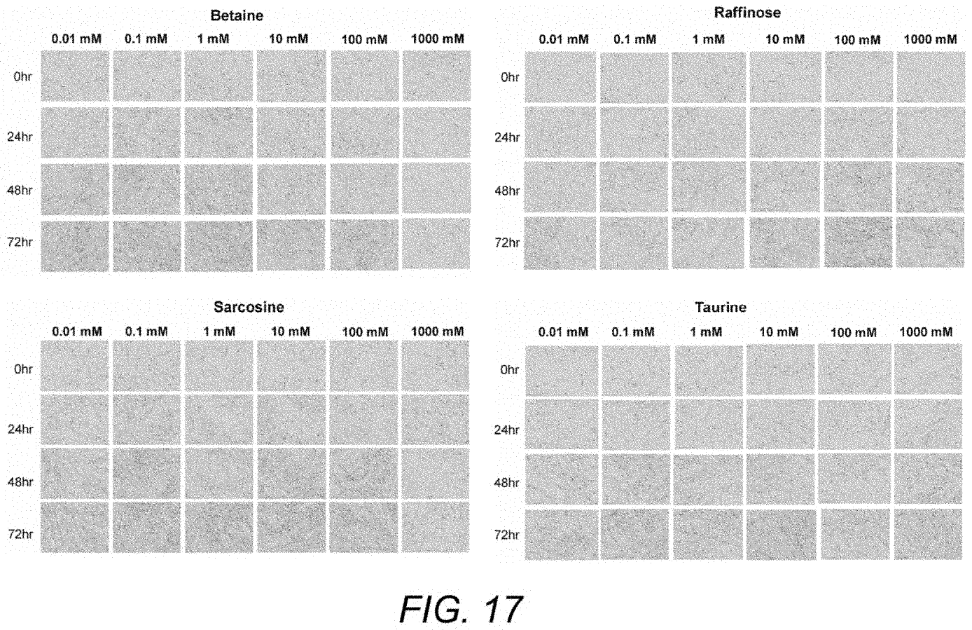

The images shown in FIG. 17 suggest that the four osmolytes at the indicated concentrations even at the elevated concentrations (1 M) are non-cytotoxic to the HCSFs.

While certain embodiments of the disclosed subject matter have been illustrated and described, it will be clear that the disclosure is not limited to the embodiments described herein. Numerous modifications, changes, variations, substitutions and equivalents are not precluded.

1. Use of an osmolyte in the manufacture of a medicament for the treatment of ocular disorders.

2. The use according to claim 1, wherein the osmolyte is selected from a group consisting of betaine, raffmose, sarcosine, taurine and/or any pharmaceutically acceptable derivatives thereof.

3. The use according to claim 1, wherein the osmolyte is a combination of taurine and sarcosine.

4. The use according to claim 1, wherein the osmolyte is selected from a group consisting of taurine and/or any pharmaceutically acceptable derivatives thereof.

5. The use according to claim 1, wherein the osmolyte has a concentration of 0.01 mM to 1,000 mM.

6. The use according to claim 1, wherein the osmolyte has a concentration of 100 mM to 500 mM.

7. The use according to claim 1, wherein the osmolyte has a concentration of 200 mM and 320 mM.

8. The use according to claim 1, wherein the medicament is in a dosage form of drops, ointments, gels and/or injections.

9. The use according to claim 1, wherein the medicament is administrated for at least 24 hours to 12 months.

10. The use according to claim 1, wherein the medicament is administrated for at least 24 hours, 48 hours, 72 hours, 1 week, 2 weeks, 4 weeks, 2 months, 4 months, 6 months, 9 months or 12 months.

11. The use according to claim 1, wherein the osmolyte inhibits amyloid fibrillation and dissolves amyloid fibrils.

12. The use according to claim 1 wherein the ocular disorders are Transforming Growth Factor-Beta Induced (TGFBI) corneal dystrophies.

13. The use according to claim 12, wherein the TGFBI corneal dystrophies are Bowman's layer corneal dystrophies and stromal corneal dystrophies.

14. The use according to claim 13, wherein the Bowman's layer corneal dystrophies are Reis-Buckler corneal dystrophy (RBCD) and Thiel-Behnke comeal dystrophy (TBCD).

15. The use according to claim 13, wherein the stromal corneal dystrophies are lattice corneal dystrophies (LCD), Granular Comeal Dystrophies Type I (GCD1) and Type H (GCD2).
